(19) United States
(12) Patent Application Publication
(10) Pub. No.: US 2012/0302759 A1
(43) Pub. Date: Nov. 29, 2012

(54) COMBRETASTATIN DERIVATIVE PREPARATION METHOD

(75) Inventors: Pascal BESSE, Paris (FR); Eric DIDIER, Paris (FR); Nicolas TREMAUDEUX, Paris (FR)

(73) Assignee: C/O SANOFI, Paris (FR)

(21) Appl. No.: 13/487,606

(22) Filed: Jun. 4, 2012

Related U.S. Application Data
(63) Continuation of application No. PCT/FR2010/052592, filed on Dec. 2, 2010.

(30) Foreign Application Priority Data
Dec. 3, 2009 (FR) 09/05837

(51) Int. Cl. C07C 231/12; C07C 271/20; C07D 309/12; C07D 263/06

(52) U.S. Cl. 548/215; 564/194; 560/29; 549/419; 558/275

(57) ABSTRACT

The invention relates to a method for preparing a combretastatin derivative (I) or (II), said method including the following steps: triaryl(3,4,5-trimethoxybenzyl)phosphonium halide P₃ (III), wherein Ar denotes an aryl group selected from among phenyl or thienyl, is reacted with P₂ having formula (IV) or P′₂ having formula (V) so as to respectively obtain the compound P₄ or P′₄, which have formulas (VI) and (VII), respectively; then, during a step for deprotection in the presence of an acid and/or a base, the compound having P₄ or P′₄ leads, after an optional purification step, to the compound having formula (I) or (II).

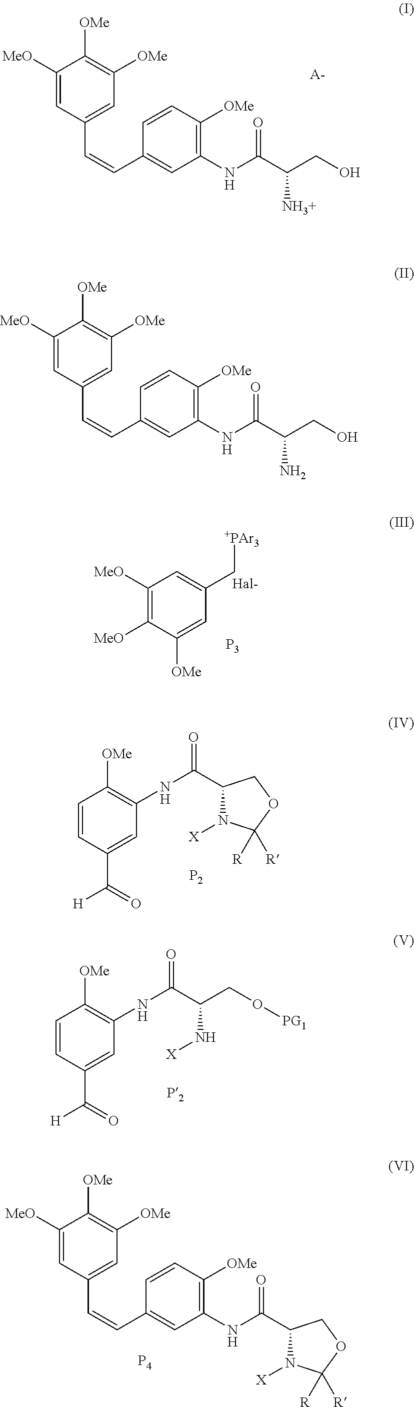

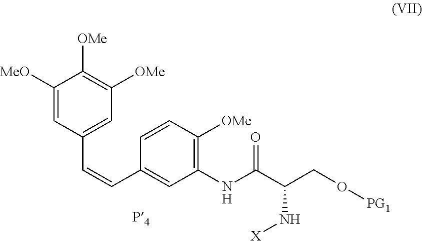

### BACKGROUND

The compounds (I) and (II) belong to the family of the combretastatin derivatives or stilbene derivatives which are anticancer compounds. They are described in Applications EP 0 731 085, EP 1 264 821, EP 1 068 870 and EP 1 407 784. The preparation of these derivatives is based, in one of the stages, on the formation of the C═C double bond. In this stage, two isomers Z and E may be formed but only the Z isomer

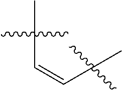

exhibits a truly effective anticancer activity. The preparation process should thus result in a high Z/E ratio.

The Applicant Company has developed an alternative process for the preparation of the compounds (I) and (II) which is based on the use of the intermediates P₂ or P′₂ described below. This process exhibits the advantage of eliminating the stage during which a cytotoxic intermediate is formed. This alternative process thus exhibits fewer stages comprising toxic compounds, which makes it easier to manage it from an industrial viewpoint.

The papers J. Fluor Chem., 2003, 123, 101-108, and Synlett, 2006, 18, 2977, describe the preparation of combretastatins using one of the stages of the Wittig reaction. The Wittig reaction is also envisaged in U.S. Pat. No. 7,265,136 and in International Applications WO 03/084919 and WO 2009/118474.

### BRIEF DESCRIPTION OF THE. INVENTION

The present patent application relates to a process for the preparation of a combretastatin derivative of formula (I) or (II):

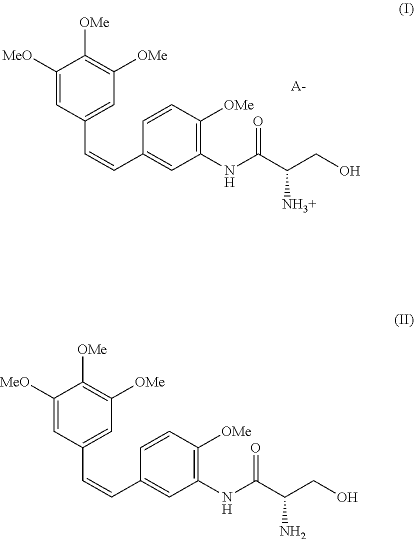

wherein A⁻ denotes an anion associated with an acid AH. More particularly, A denotes Cl⁻.

The invention relates to a process for the preparation of a combretastatin derivative of formula (I) or (II):

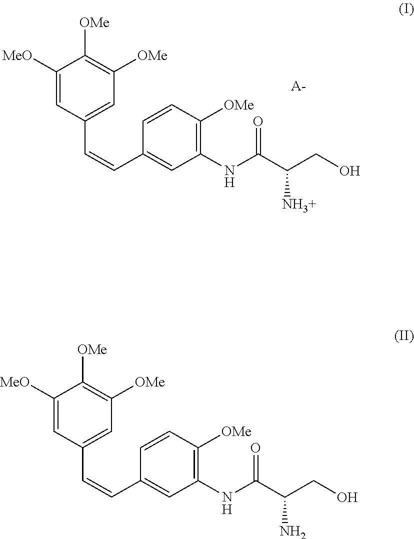

wherein A⁻ denotes an anion associated with an acid AH comprising the following steps:

reacting, in the presence of a base, a halide of triaryl(3,4,5-trimethoxybenzyl)phosphonium P₃

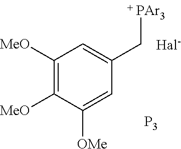

in which Ar denotes an aryl group chosen from phenyl or thienyl, optionally substituted by a (C₁-C₄)alkyl, (C₁-C₄)alkoxy or halogen group,

with P₂ of formula:

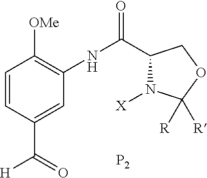

wherein each of R and R′ represent a (C₁-C₄)alkyl group, or R represents a phenyl group optionally substituted by a (C₁-C₄)alkoxy group and R′ represents a hydrogen atom, or R and R′ form, together with the carbon atom to which they are connected, a (C₃-C₇)cycloalkyl group;

or with P′₂ of formula:

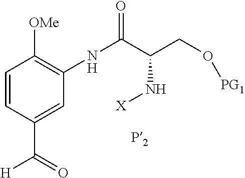

wherein PG₁ is a protective group for the alcohol functional group, and X is boc, Fmoc or CBZ,

such that compound P₄ or P′₄ is obtained, respectively

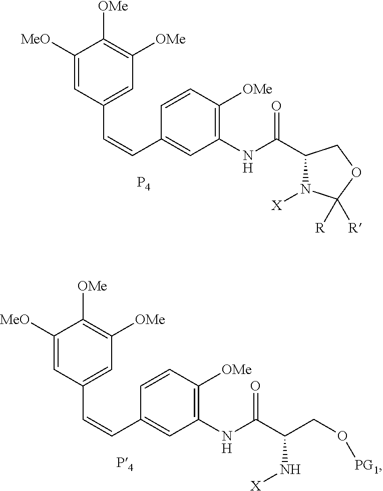

deprotecting the compound of formula P₄ or P′₄ in the presence of an acid and/or of a base, to produce the compound of formula (I) or (II); and

optionally purifying the compound of formula (I) or (II).

The invention also relates to a compound of formula P₂:

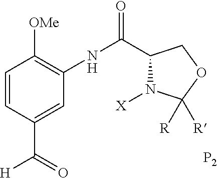

wherein each of R and R′ represent a (C₁-C₄)alkyl group, or R represents a phenyl group optionally substituted by a (C₁-C₄)alkoxy group and R′ represents a hydrogen atom, or R and R′ form, together with the carbon atom to which they are connected, a (C₃-C₇)cycloalkyl group, and X is boc, Fmoc or CBZ.

The invention also relates to the compound of formula P′₂:

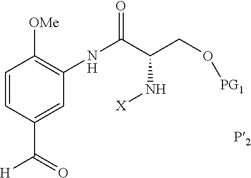

wherein PG₁ is a protective group for the alcohol functional group and X is boc, Fmoc or CBZ.

R and R′ can, for example, both represent a methyl (Me) group or can form, together with the carbon atom to which they are connected, a cyclohexyl group. X can, for example, represent boc. PG₁ can, for example, represent one of the following protective groups: THP (tetrahydropyran), MEM (methoxyethoxymethyl), boc, trityl or acetyl (Ac). Ar can represent the phenyl or thienyl group, optionally substituted by a (C₁-C₄)alkyl or (C₁-C₄)alkoxy group. A⁻ can denote Cl⁻.

The invention also relates to the use of one of the two compounds P₂ and P′₂ as an intermediate in the preparation of a compound of formula (I) or (II).

The invention also relates to the use of one of the two compounds P₄ and P′₄ as an intermediate in the preparation of a compound of formula (I) or (II).

### DETAILED DESCRIPTION OF THE INVENTION

The general Scheme 1 describes stages (i) to (iv) of the process:

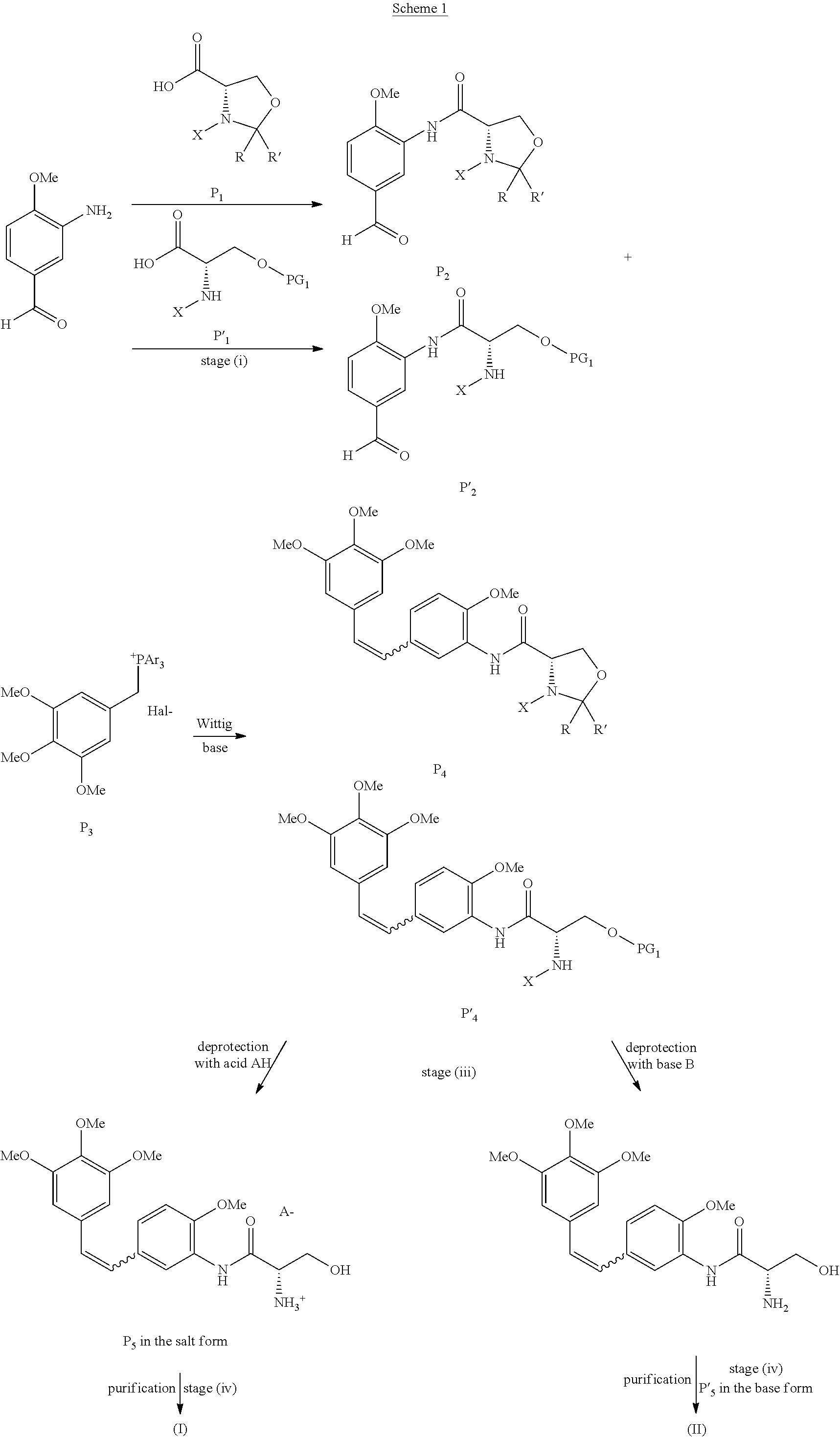

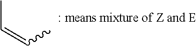

Stage (i): coupling a 3-amino-4-methoxybenzaldehyde and a protected serine of formula:
P₁, wherein each of R and R′ represents a (C₁-C₄)alkyl group, or R represents a phenyl group optionally substituted by a (C₁-C₄)alkoxy group, for example methoxy, and R′ represents a hydrogen atom, or R and R′ form, together with the carbon atom to which they are connected, a (C₃-C₇)cycloalkyl group,
or with formula P′₁, wherein PG₁ is a protective group for the alcohol functional group. On conclusion of this coupling, P₂ or P′₂ respectively is obtained
X represents boc, Fmoc or CBZ.

P₁ can more particularly be one of the following compounds:

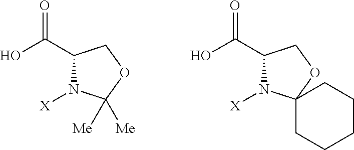

and in particular those for which X=boc (for example, compound 8 of Synthesis, 2006, 8, 1289-1294, for which R═R′=Me).

P′₁ can more particularly be one of the following compounds:

X=boc, PG₁=THP: see compound 13a of Ex. 13 of WO 06042215;

X═PG₁=boc: Justus Liebigs Annalen der Chemie, 1971, 743, 57-68;

X=Fmoc, PG₁=Ac: commercial compound of formula:

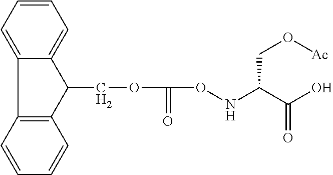

PG₁ is a protective group for the alcohol functional group. boc, Fmoc and CBZ respectively denote the tert-butoxycarbonyl, 9-fluorenylmethoxycarbonyl and benzyloxycarbonyl groups. A protective group is a chemical entity which is introduced onto a molecule during a “protection” stage by modification of a chemical group, making it possible to improve the chemoselectivity of a reaction by preventing undesirable side reactions at the said chemical group, and which is released during a subsequent “deprotection” stage. PG₁ can, for example, be THP (tetrahydropyran), MEM (methoxyethoxymethyl), boc, trityl, or acetyl (Ac).

The coupling (amidation) is advantageously carried out in the presence of an acid activator. The term “acid activator” denotes a compound having the role of rendering the acid functional group —COOH of P₁ or P′₁ more reactive for the purpose of promoting the formation of an amide bond. Reference may be made, for further details with regard to acid activators, to the review ChemFiles, Vol. 7, No. 2, page 3, edited by Aldrich Chemical, or else to Tetrahedron Report, No. 672, 2004, 60, 2447-2467, “Recent development of peptide coupling reagents in organic synthesis”. EDCI (1-(3-dimethylaminopropyl)-3-ethylcarbodiimide) chloride), DCC (dicyclohexylcarbodiimide), TOTU (O-[ethoxycarbonyl]cyanomethyleneamino)-N,N,N′,N′-tetramethyluronium tetrafluoroborate), HBTU (O-(benzotriazol-1-yl)-N,N,N′,N′-tetramethyluronium hexafluorophosphate) and N,N-carbonyldiimidazole are examples of acid activators or the anhydride of propanephosphonic acid (T3P). In the presence of the acid activator, an isolable or non-isolable intermediate may be formed which comprises an activated acid functional group of the form —COZ; for example, in the case of pivaloyl chloride, Z represents —OtBu.

The coupling can be carried out in a solvent, such as, for example, a chlorinated solvent, for example dichloromethane (DCM), an ether, for example. THF, or an aromatic solvent, for example toluene, at a temperature which can be between 0° C. and 20° C.

Stage (ii): Wittig reaction between P₂ or P′₂ and triaryl(3,4,5-trimethoxybenzyl)phosphonium halide P₃, resulting respectively in P₄ or P′₄. In P₃, Ar denotes an aryl group chosen from phenyl or thienyl, optionally substituted by a (C₁-C₄)alkyl or (C₁-C₄)alkoxy group.

The Wittig reaction is carried out in a solvent in the presence of base. P₃ is obtained by the reaction of 3,4,5-trimethoxybenzyl halide with the corresponding triarylphosphine PAr₃. Use is preferably made of a chloride or a bromide. An example of P₃ is triphenyl(3,4,5-trimethoxybenzyl)phosphonium chloride, which is described on p. 102 of J. Fluor. Chem., 2003, 123, 101-108, or else its bromide equivalent, which is described on pp. 15-16 of WO 02/06279.

The solvent of this reaction can, for example, be toluene, THF, dimethylformamide (DMF), chloroform, DCM, trifluorotoluene, a mixture of these solvents or an aqueous two-phase mixture, such as, for example, the chloroform/water mixture.

The base which is used is preferably a strong base, such as, for example, NaHMDS (sodium bis(trimethylsilyl)amide; CAS [1070-89-9]), KHMDS (potassium bis(trimethylsilyl)amide; CAS [40949-94-8]), sodium methoxide, sodium amide or sodium hydroxide. The base can be brought together with a phosphonium salt P₃ and then the aldehyde P₂ or P′₂ can be run onto the phosphonium salt P₃, which will have been brought into contact beforehand with the base. According to a preferred alternative form which makes it possible to obtain a higher yield of P₄ or P′₄, the base is run onto the mixture formed by the aldehyde and the phosphonium salt.

The Wittig reaction can be carried out at a temperature generally of between 0° C. and the reflux temperature of the solvent.

Stage (iii): the deprotection of P₄ or P′₄ is carried out in one or more stages and under conditions which depend on the nature of the protective groups X and, if appropriate, PG₁. A person skilled in the art may refer to “Greene's Protective Groups in Organic Synthesis”, 4th edition, ISBN 978-0-471-69754-1, to find, if appropriate, these conditions.

Thus, for some protective groups (for example, compound P₄ with X=boc), the deprotection can be carried out in the presence of an organic or inorganic acid AH. In this case, the deprotection results in the compound P₅ in the salt form. For other protective groups, the deprotection can be carried out in the presence of an organic or inorganic base B. In this case, the deprotection results in the compound P′₅ in the base form. The temperature of the deprotection reaction is preferably between 0° C. and 50° C. The acid can be a strong acid, such as, for example, hydrochloric acid, which results in the hydrochloride. The base can be, for example, sodium hydroxide. It is also possible to combine an acid treatment and a basic treatment, in particular for P′₄, which comprises two different protective groups X and PG₁.

Stage (iv): if necessary, the Z isomer is separated from the E isomer by any purification technique known in organic synthesis. It can be purification by recrystallization, using as solvent a mixture comprising an alcohol and a ketone or an ester and more particularly the methyl ethyl ketone (MEK)/water mixture.

Stage (iii) or, if appropriate, (iv) can optionally be followed by an additional stage consisting in converting:
by addition of an acid, a combretastatin in the base form (for example (II)) into combretastatin in the salt form (for example (I)) or, by addition of a base, the combretastatin in the salt form (for example (I)) into combretastatin in the base form (form example (II)).

### Intermediates P₁ and P′₁

P₁ is obtained according to Scheme 2 by reaction of a ketone and of a derivative of L-serine, the amine functional group of which has been protected with X.

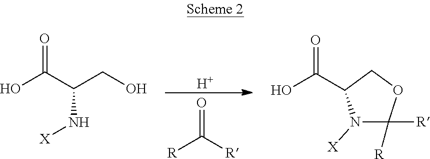

P′₁ is obtained by protection of the —OH functional group of a derivative of L-serine, the amine functional group of which has been protected with X.

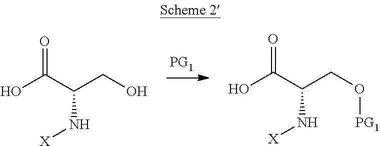

The derivative of L-serine of Schemes 2 and 2′ can be commercially available (for example, N-boc-L-serine) or readily accessible using at least one chemical reaction known to a person skilled in the art (similar, for example, to that which makes it possible to prepare N-boc-L-serine).

### EXAMPLES

### Example 1

### Preparation of the Hydrochloride of the Compound (II)

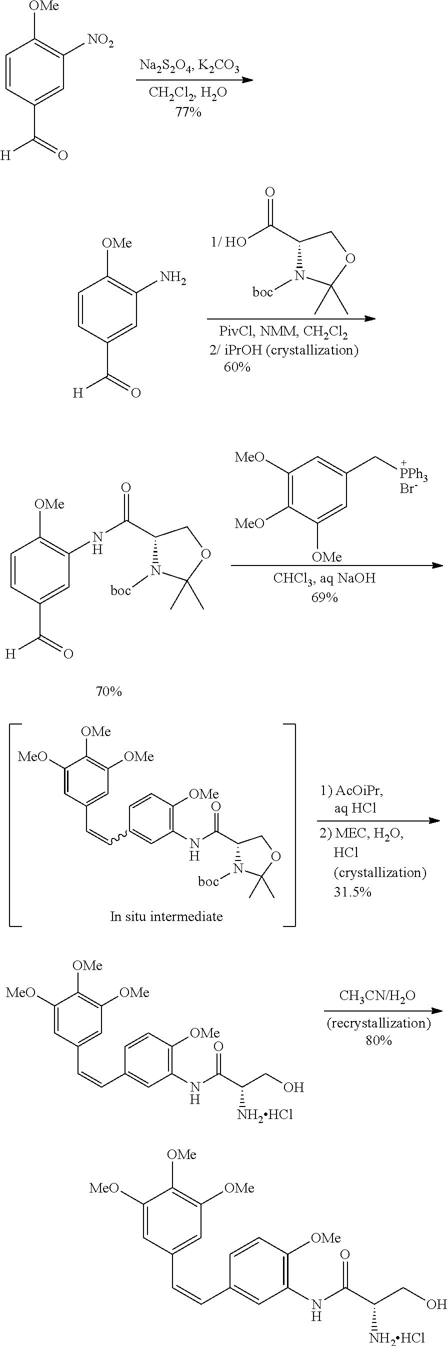

3-Amino-4-methoxybenzaldehyde was obtained by reduction of the corresponding nitro compound according to Tetrahedron Letters, 1993, 34(46), 7445-1446.

### Preparation of P₂ (Stage (i))

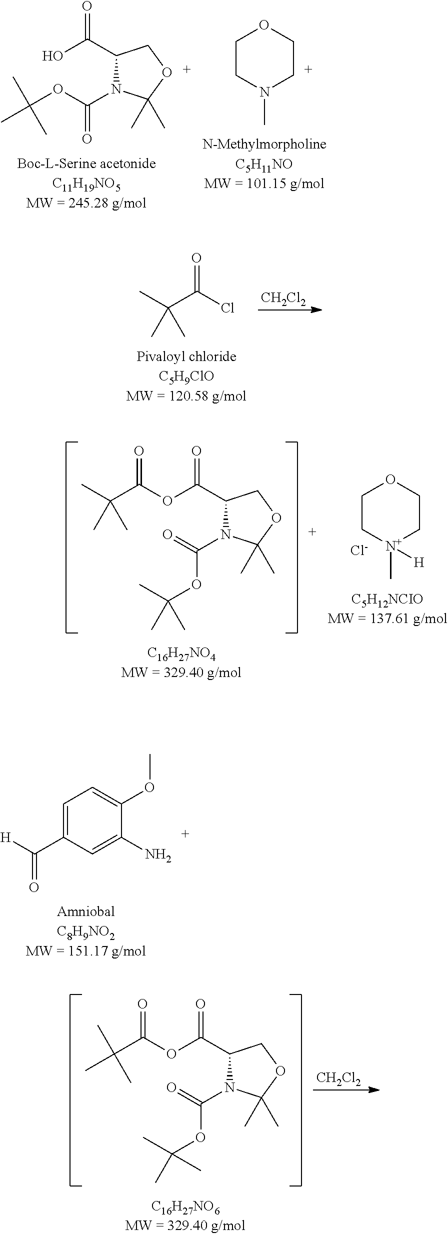

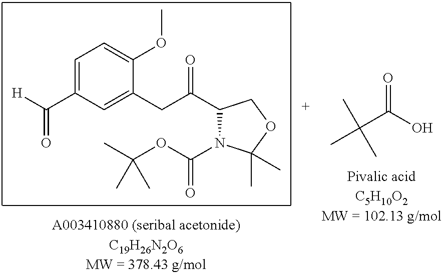

Before being used, the reactor was freed with DCM, dried under vacuum and purged by flushing with nitrogen for 15 to 30 min, the Erlenmeyer flask is rinsed with amylene-stabilized DCM and then dried under nitrogen. The reactor was charged with 95 ml of DCM and 34.0 g of boc-L-serine acetonide, cooled to 4-10° C. and 14.3 g of N-methylmorpholine were added using a dropping funnel while maintaining the temperature at 4-10° C. The dropping funnel was rinsed with 2.5 ml of DCM. 17.1 g of pivaloyl chloride were added using a dropping funnel while maintaining the temperature at 4-10° C. and the dropping funnel was rinsed with 2.5 ml of DCM. The mixture is kept stirred at 4-10° C. for 2 h.

A solution of aminobal (20.0 g) in DCM (95 ml) was prepared with stirring and this solution was added to the reactor while maintaining the temperature at 4-10° C. The mixture was subsequently heated to 20° C. over 1 h and was kept stirred at 20° C. for a minimum of 16 h. 100 ml of demineralized water was added to the reactor at 20-25° C. and the mixture was left stirring for 20 min and separated by settling. The lower organic phase comprising the product and the upper phase (predominantly aqueous) were withdrawn. The organic phase comprising the product was again charged to the reactor. 140 ml of a 1.0 N aqueous sodium hydroxide solution were added. The mixture was kept stirred at 20-25° C. for approximately 20 min and then allowed to separate by settling. The lower organic phase comprising the product was withdrawn. The organic phase comprising the product was again charged to the reactor. 100 ml of demineralized water were added. The mixture was kept stirred at 20-25° C. for approximately 20 min and then allowed to separate by settling. The lower organic phase comprising the product was withdrawn. The organic phase comprising the product was again charged to the reactor. 100 ml of isopropanol was added.

Distillation was carried out (35±5° C. in the jacket) under a residual pressure of approximately 30 mbar until a residual volume of 100 ml was present in the reactor. The temperature was adjusted to 20° C. and the mixture was left stirring at 20° C. for 3 h. The reactor was rinsed and the cake was washed twice with a total volume of 40 ml of isopropanol. The product was dried at 40° C. under a vacuum of 30 mbar. Yield of isolated product: 60%.

Wittig Reaction (Stage (ii))

A 7 L reactor was charged with 581 g of phosphonium salt (1.2 eq.), 350 g of the aldehyde from the preceding stage (1.0 eq.) and 3500 ml of CHCl₃ (intense yellow-brown solution). 1110 ml of a 1N NaOH solution (1.2 eq.) were added. The two-phase mixture was stirred vigorously and the solution became pale yellow. It was kept at approximately 20° C. 3500 ml of water were added and the mixture was stirred and separated by settling (pH of the aqueous phase 13). A 2nd washing was carried out with 3500 ml of water; the pH was then 7. Separation by settling was carried out and the yellow-orange organic phase was withdrawn (volume 4250 ml comprising 346.0 g of Z and 136.7 g of E). The Z/E ratio was 72/28 and the Z+E yield with respect to the aldehyde was 96.2%.

The solution was reintroduced into the reactor and then the CHCl₃ was distilled off under vacuum with a starting vacuum of 100 mbar and a final vacuum of 45 mbar (jacket temperature approximately 30° C.). The mixture became syrupy. The vacuum was broken and 50 ml of CHCl₃ and 2500 ml of AcOiPr were added: a fluid solution was obtained (5250 ml). The distillation was resumed at constant volume with addition of AcOiPr. Crystals (predominantly of triphenylphosphine oxide) were formed and were filtered off. The filtrate comprising the expected product was retained for use in the following stage. Z/E ratio=71/29. Z yield: 68.9%.

Deprotection in an Acid Medium (Stage (iii))

The solution from the preceding stage'(3045.9 g of solution, i.e. 343.9 g of Z and 136.9 g of E) was charged. 295.2 ml of a 12N HCl solution (4 eq., with respect to the product) were added. The two-phase mixture changes from yellow to dark red. 1800 ml of water were added, the mixture was stirred for 10 min and separated by settling, and the rich aqueous phase was withdrawn. 900 ml of water were added to the organic phase. The mixture was separated by settling and the aqueous phase was withdrawn. 3714 g of orange aqueous phase were obtained (Z/E ratio=67/33). 2700 ml of AcOiPr were added and a 10N NaOH solution was run in slowly until a pH of 10-11 was obtained. The mixture was separated by settling and the aqueous phase was withdrawn. 2700 ml of water and 11 g of NaCl were added and the mixture was vigorously stirred and then separated by settling. This whisking operation was repeated with 2700 ml of water. A yellow organic phase was recovered (2760 g) Z/E ratio=68/32. Yield: 35%.

Recrystallization (Stage (iv))

A 250 ml three-necked flask was charged with 5.27 g of the preceding product, 50 ml of water, 50 ml of AcOiPr and 1.32 ml of 30% sodium hydroxide. The mixture was stirred for 30 min. It was separated by settling and the aqueous phase (pH=10) was withdrawn. Two whisking operations were carried out with water (50 ml). After the 2nd whisking operation, the pH was 7. The organic phase was evaporated to dryness (40° C., vacuum of 60 mbar) and the residue was dried in an oven (40° C.). The solid (5.49 g) was taken up in 11.2 ml of MEC, and 1.00 ml of a 12N HCl solution (density=1.18) was added to the solution. A small amount of product was allowed to slowly crystallize. 0.36 ml of water was added and a large part of the crystallized product redissolved. 2.70 ml of MEC was then added and crystallization was again allowed to take place. The mixture was stirred at ambient temperature for 5 days. The product was obtained with a Z/E ratio=93/07. Z yield: 45%.

### Example 1a

### Preparation of the Hydrochloride of the Compound (II)

Wittig Reaction (Stage (ii))

A 500 ml reactor was charged with 44.8 g of phosphonium salt (1.2 eq.), 27 g of the aldehyde from the preceding stage (1.0 eq.) and 270 ml of CHCl₃ (intense yellow-brown solution). 85.6 ml of a 1N NaOH solution (1.2 eq.) was added. The two-phase mixture was stirred vigorously and the solution became pale yellow. It was maintained at approximately 20° C. for approximately 4 h. 270 ml of water was added and the mixture was stirred and separated by settling (pH of the aqueous phase 13). A 2nd washing operation was carried out with 270 ml of water; the pH as then 7. The mixture was separated by settling and the yellow-orange organic phase (weight 470.4 g, comprising 26.7 g of Z and 11.2 g of E) was withdrawn. The Z/E ratio was 70/30, the Z+E ratio with respect to the aldehyde was 98% and the Z yield with respect to the aldehyde was 69.0%.

The solution was reintroduced into the reactor and then a change in solvent to isopropyl acetate was carried out under reduced pressure (45 to 100 mbar at 30° C. approximately). At the end of the operation, the residual volume was adjusted to 203 ml. Crystals were formed, which were filtered off and washed with isopropyl acetate. The filtrate, comprising the reaction product, was used as is in the following stage. Z/E ratio=70/30. Z yield: 69.0%.

Deprotection in Acidic Medium (Stage (iii))

The solution from the preceding stage (248.0 g of solution, i.e. 26.7 g of Z and 11.2 g of E) was charged to a 500 ml reactor. 23.3 ml of a 12N HCl solution (4 eq. with respect to the product) were added. The two-phase mixture changed from yellow to dark red. The mixture was kept stirred at 20° C. for approximately 5 h. 137 ml of water were added, the mixture was stirred for 10 min and separated by settling, and the rich aqueous phase was withdrawn. 69 ml of water were added to the organic phase. The mixture was separated by settling and the aqueous phase was withdrawn. 283.6 g of orange aqueous phase were obtained (Z/E ratio=66/34). 206 ml of AcOiPr were added and a 10N NaOH solution was slowly added until a pH of 10-11 was obtained. The mixture was separated by settling and the aqueous phase was withdrawn. 206 ml of water and 2.1 g of NaCl were added and the mixture was vigorously stirred and then separated by settling. This operation was repeated a second time. A yellow organic phase was recovered and was brought to dryness (35.0 g, Z/E ratio=66/34). This residue was taken up in 108.3 g of MEC. A solution was obtained. 5.82 ml of 12N HCl and 2.75 ml of water were successively added. Initiation was subsequently carried out by the addition of 75 mg of pure Z isomer. The mixture was kept stirred at 20° C. for 24 h and then the slurry obtained was filtered. The cake was pulled as dry as possible and then dried in an oven (50° C., 60 mbar). 7.15 g of a fine beige powder was thus obtained: Z yield: 31.5%, Z/E ratio=95.9/4.1.

Recrystallization (Stage (iv))

A 5 ml round-bottomed flaks was charged with 488 mg of compound (I) (Z/E=93.5/6.5), 0.115 ml of water and 268 ml of acetonitrile. The mixture was heated to 35° C., stirred until a solution was obtained and then cooled to 20° C. Initiation was carried out at this temperature with 3 mg of the pure Z isomer. The mixture was kept stirred for 30 min and then 3.44 ml of acetonitrile were added over approximately 2 h. The mixture was subsequently kept stirred at 20° C. for 18 h and filtered. The cake obtained was dried in an oven (50° C., 60 mbar). 367 mg of the expected product were thus obtained with a Z/E ratio of 99.65/0.35, i.e. a yield of 80%.

1-12. (canceled)

13. A process for preparing a combretastatin derivative of formula (I) or (II): 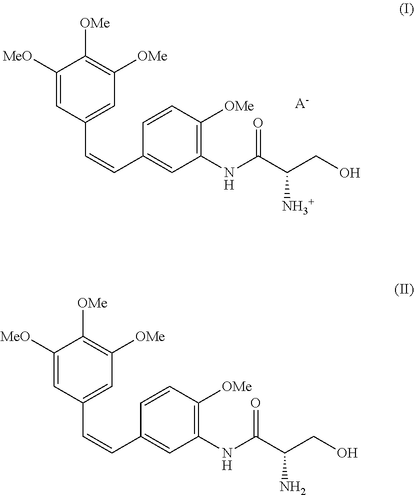
wherein A⁻ is an anion associated with an acid AH, comprising the steps of:
reacting, under suitable conditions, a triaryl(3,4,5-trimethoxybenzyl)phosphonium halide P₃ 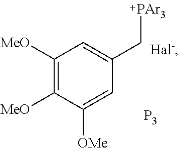
wherein Ar denotes an aryl group chosen from phenyl or thienyl, optionally substituted by a (C₁-C₄)alkyl, (C₁-C₄)alkoxy or halogen group, wherein suitable conditions comprise the presence of a base,
with P₂ of formula: 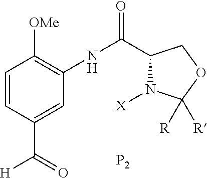
wherein each R and R′ represents a (C₁-C₄)alkyl group, or R represents a phenyl group optionally substituted by a (C₁-C₄)alkoxy group and R′ represents a hydrogen atom, or R and R′ form, together with the carbon atom to which they are connected, a (C₃-C₇)cycloalkyl group;
or with P′₂ of formula: 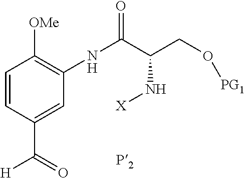
wherein PG₁ is a protective group,
X is boc, Fmoc, or CBZ,
such that compound P₄ or P′₄ is obtained, respectively 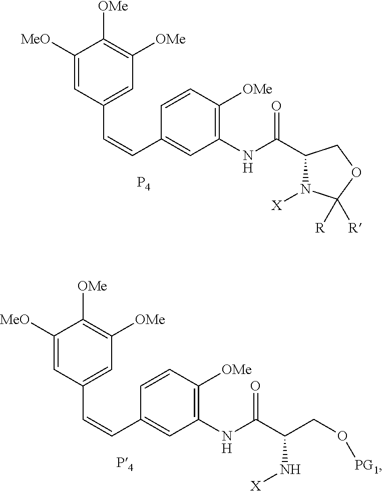
deprotecting in the compound of formula P₄ or P′₄ in the presence of an acid and/or of a base to produce the compound of formula (I) or (II); and
optionally purifying the compound of formula (I) or (II).

14. The process of claim 13, wherein R and R′ both represent a methyl group or form, together with the carbon atom to which they are connected, a cyclohexyl group.

15. The process of claim 13, wherein X is boc.

16. The process of claim 14, wherein X is boc.

17. The process of claim 13, wherein PG₁ is selected from the group consisting of: THP (tetrahydropyran), MEM (methoxyethoxymethyl), boc, trityl or acetyl (Ac).

18. The process of claim 14, wherein PG₁ is selected from the group consisting of: THP (tetrahydropyran), MEM (methoxyethoxymethyl), boc, trityl or acetyl (Ac).

19. The process of claim 15, wherein PG₁ is selected from the group consisting of: THP (tetrahydropyran), MEM (methoxyethoxymethyl), boc, trityl or acetyl (Ac).

20. The process of claim 14, wherein Ar is s the phenyl or thienyl group, optionally substituted by a (C₁-C₄)alkyl or (C₁-C₄)alkoxy group.

21. The process of claim 15, wherein Ar is s the phenyl or thienyl group, optionally substituted by a (C₁-C₄)alkyl or (C₁-C₄)alkoxy group.

22. The process of claim 17, wherein Ar is s the phenyl or thienyl group, optionally substituted by a (C₁-C₄)alkyl or (C₁-C₄)alkoxy group.

23. The process of claim 13, wherein A⁻ is Cl⁻.

24. The process of claim 14, wherein A⁻ is Cl⁻.

25. The process of claim 15, wherein A⁻ is Cl⁻.

26. The process of claim 17, wherein A⁻ is Cl⁻.

27. The process of claim 20, wherein A⁻ is Cl⁻.

28. The process of claim 13, wherein P₃ is reacted with P₂.

29. A compound of formula P₂: 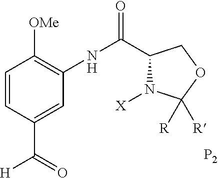
wherein each of R and R′ represent a (C₁-C₄)alkyl group or R represents a phenyl group optionally substituted by a (C₁-C₄)alkoxy group and R′ represents a hydrogen atom, or R and R′ form, together with the carbon atom to which they are connected, a (C₃-C₇)cycloalkyl group; and X is boc, Fmoc or CBZ.

30. The compound of claim 29, wherein X is boc.

31. The compound of claim 29, wherein R and R′ both represent a methyl group or R and R′ form, together with the carbon atom to which they are connected, a cyclohexyl group.

32. The compound of formula P′₂: 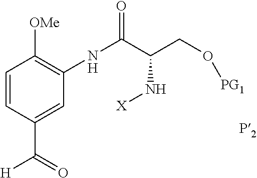
wherein PG₁ is a protective group and X is boc, Fmoc or CBZ.

33. The compound of claim 32, wherein PG₁ is selected from the group consisting of THP (tetrahydropyran), MEM (methoxyethoxymethyl), boc, trityl, and acetyl (Ac).
